(19) United States
(12) Patent Application Publication
(10) Pub. No.: US 2004/0215189 A1
(43) Pub. Date: Oct. 28, 2004

(54) Pedicle augmenter for vertebral body reconstruction

(76) Inventor: Kung-Chia Li, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/420,798

(22) Filed: Apr. 23, 2003

(51) Int. Cl. A61B 17/56

(52) U.S. Cl. 606/57

(57) ABSTRACT

A pedicle augmenter is provided for reconstruction of a vertebra subject to loss of bone mass or fracture, and is to be planted into the vertebra from a pedicle of the vertebra for helping maintain or restore normal position and size of the vertebra. Through the vertebral pedicle, a device, fit the pedicle anatomy, was created to reconstruct the fractured or collapsed vertebral body. The device is a rectangular solid with smooth edge and the first-end was with ellipsoid shape. The device has porous surface. The internal screw file for the instrument connection was createdfrom a second-end towars to a first-end. The fractured body was reduced and maintained by such device until the bone growth and fracture union.

### BACKGROUND OF THE INVENTION

1. Field of the Invention

The present invention relates to a pedicle augmenter for vertebral body reconstruction, more particularly, which is used for treatment of vertebral burst fractures, and vertebral compression fractures.

2. Brief Description of the Prior Art

Vertebral body fractures including acute burst fracture or chronic compression fracture with kyphosis were treated in various ways. Acute burst fracture can be treated conservatively, surgical posterior instrumentation, or even anterior bone graft and posterior instrumentation. However, conservative treatment is limited to those with neural intact, non-osteoporosis, good compliance to wear brace and less kyphosis. Posterior instrumentation alone is easy and simple but may lead to 20% implant failure. However, combining anterior and posterior approaches proved to have mechanical advantages, but surgery itself is a suffering to elder patients. The best way to those not good for conservative treatment will be posterior approach with functions of anterior approach simultaneously.

In chronic osteoporotic compression fractures may induce lots of morbilities including chronic back pain, GI dysfunction, decreased pulmonary vital capacity, and feasible new compression fractures. The various methods including surgical instrumentation, vertebral augmentation with cement, kyphoplasty etc. The techniques improved and results become better recently. However, instrumentation may induce decreased spine ROM, implant failure, and adjacent segment disease. The cement either in augmentation or kyphoplasty may induce cement disease. The kyphosis correction is not perfect in kyphoplasty especially the chronic cases with anterior partial or fully fusion. The pedicle augmenter tried to correct the collapsed vertebral body and lead to union with bone graft either combined short posterior instrumentation or cement techniques.

### SUMMARY OF THE INVENTION

It is a main object of the present invention to provide a pedicle augmenter, which can be planted into an osteoporosis-effected vertebra from a pedicle of the vertebra, for helping maintain or restore normal position and size of the vertebra.

Through the vertebral pedicle, a device, fit the pedicle anatomy, was created to reconstruct the fractured or collapsed vertebral body. The device is a rectangular solid with smooth edge and the first-end was with ellipsoid shape. The device has porous surface. The internal screw file for the instrument connection was created from a second-end towars to a first-end. The fractured body was reduced and maintained by such device until the bone growth and fracture union.

### BRIEF DESCRIPTION OF THE DRAWINGS

FIG. 1 is a perspective view of the pedicle augmenter for vertebral body reconstruction according to the present invention,

FIG. 2 is a cross-sectional view of the pedicle augmenter for vertebral body reconstruction in use according to the present invention; and

FIG. 3 is a cross-sectional view of the pedicle augmenter for vertebral body reconstruction in use according to the second embodiment.

### DETAILED DESCRIPTION OF THE PREFERRED EMBODIMENTS

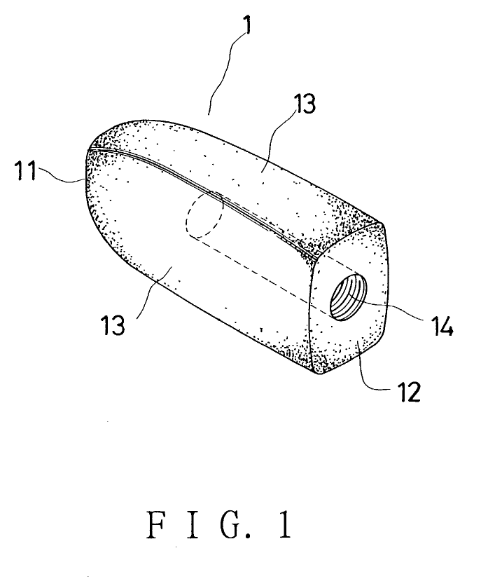

Referring to FIG. 1, a preferred embodiment of a pedicle augmenter 1 for vertebral body reconstruction in the present invention tapers off from a second end 12 towards a first end 11 near the first-end part thereof, and is formed with smooth shape on four long sides thereof, and a screw hole 14 extending from the second end 12 towards to the first end 11; the first-end having ellipsoid shape. The present pedicle augmenter 1 is provided particularly for reconstruction of a collapsed vertebra that is still relatively complete in structure, and doesn't need adjustment. In a second embodiment of the present invention, the pedicle augmenter 1 for vertebral body reconstruction is formed porous outer surface instead of smooth one; thus, the outer surface of the present pedicle augmenter I is porous for bone-ingrowth.

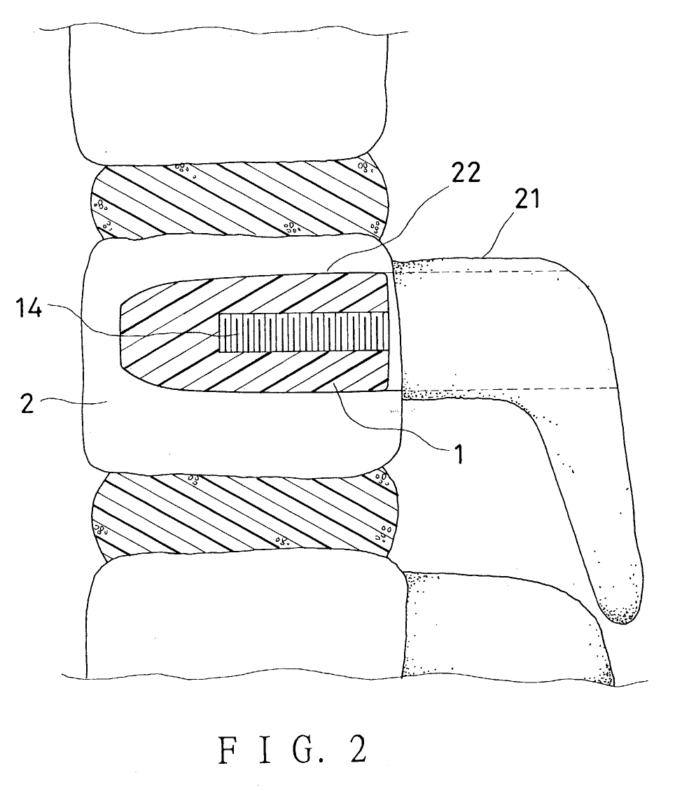

Referring to FIG. 2, to plant the pedicle augmenter 1 into a vertebra 2, first a planting hole 22 is formed, which extends from a pedicle 21 of the vertebra 2 to the main body of the vertebra 2, i.e. the vertebral body, and which is substantially as big as the pedicle augmenter 1. Second, a planting tool (not shown) is screwed into the screw hole 14 of the pedicle augmenter 1 at front end, and then the pedicle augmenter 1 is planted into the planting hole 22 in the vertebral body by means of the planting tool, and the planting tool is removed from the pedicle augmenter 1. Finally, other bone of the same patient or artificial bone is injected into the vertebra 2 so that the pedicle augmenter 1 is fixedly disposed in the vertebra 2.

The pedicle augmenter 1 is made according to the vertical cross-section and the height of the pedicle 21 of the vertebra 2; the vertical cross-section of the pedicle 21 is close to a rectangle, and somewhat oval, and the distance from an upper end to a lower end of the pedicle 21 is larger than that from a rear end to a front end; the proportion of the height of pedicle 21 to the height of the vertebra 2 is 50% to 60%. Therefore, the pedicle augmenter 1 is made with a size near to the pedicle 21 so that at least 50% to 60% of the vertebra 2 can be reconstructed, and in addition, 70% to 80% of the vertebra 2 can be reconstructed when the pedicle augmenter 1 is combined with the rest of a collapsed vertebra 2.

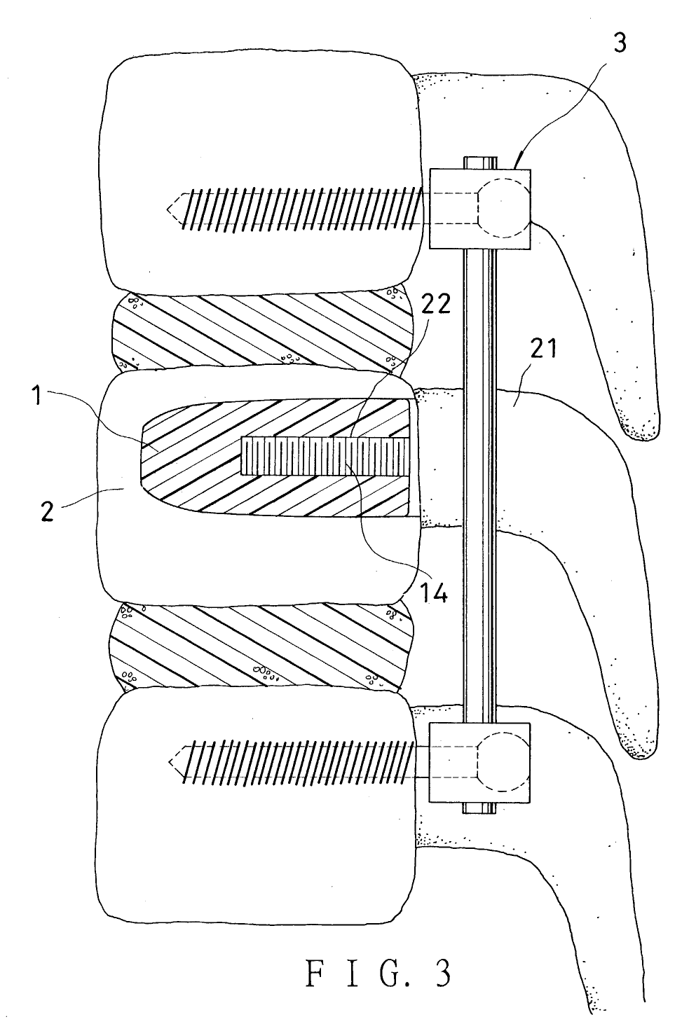

Referring to FIG. 3, if it is necessary, a pedicle screw system 3 can be applied to both of vertebras adjacent to the vertebra 2 for helping increase stability of the spine after planting of the pedicle augmenter 1 of the present invention.

From the above description, it can be easily understood that the pedicle augmenter of the present invention has advantages as followings:

1. The pedicle augmenter is planted into an collapsed vertebra from a pedicle of the vertebra instead of via the thoracic or abdominal cavity of the patient therefore the operation is relatively safe as compared with the last conventional ways as described in the Background, and mechanical strength of the vertebra is increased.

2. An collapsed vertebra, of which the size is reduced due to loss of bone mass, can be enlarged when the pedicle augmenter is planted into it. Furthermore, an original normal width and height of the collapsed vertebra can be maintained by means of injecting harvested autogenous bone graft or artificial bone into the vertebra.

3. Because the pedicle augmenter is formed with a porous outer surface, stability of connection of bone mass of a vertebra can be increased when the pedicle augmenter joined to the vertebra, and the pedicle augmenter can help the vertebra healing. And, the pedicle augmenter can be used alone on a collapsed vertebra without the need for operation being done on other healthy vertebras near to the collapsed vertebra.

What is claimed is:

1. A pedicle augmenter for vertebral body reconstruction, throught a vertebral pedicle, a device, fit a pedicle anatomy, being created to reconstruct a fractured or collapsed vertebral body; the device being a rectangular solid with smooth edge and a first-end having ellipsoid shape; an internal screw file for the instrument connection being created from a second-end towars to the first-end; the fractured body being reduced and maintained by such device until the bone growth and fracture union.

2. The pedicle augmenter for vertebral body reconstruction as claimed in claim 1 having porous surface.
